(19) United States
(12) Patent Application Publication
(10) Pub. No.: US 2025/0098937 A1
(43) Pub. Date: Mar. 27, 2025

(54) AUTONOMOUS LUMEN CENTERING OF ENDOBRONCHIAL ACCESS DEVICES

(71) Applicant: Covidien LP, Mansfield, MA (US)

(72) Inventors: Paul M. Loschak, Somerville, MA (US); Ofer Barasofsky, Tel Mond (IL); Ariel Birenbaum, Raanana (IL); Guy Alexandroni, Yehud-Monosson (IL); Nathan J. Knutson, Long Lake, MN (US); Dany Junio, Brookline, MA (US); John W. Komp, Dillon, CO (US)

(73) Assignee: Covidien LP, Mansfield, MA (US)

(21) Appl. No.: 18/810,354

(22) Filed: Aug. 20, 2024

Related U.S. Application Data
(60) Provisional application No. 63/540,513, filed on Sep. 26, 2023.

(51) Int. Cl. A61B 1/00; A61B 1/267

(52) U.S. Cl. CPC A61B 1/00006; A61B 1/0005; A61B 1/00057; A61B 1/267

(57) ABSTRACT

A system for performing a surgical procedure includes an extended working channel (EWC) having a drive mechanism, a catheter having a camera and an electromagnetic sensor, and a workstation including a memory storing instructions, which when executed by a processor cause the processor to determine a location and an orientation of a distal end of the EWC, receive real-time images of the patient's anatomy from the camera, identify a centerpoint of a lumen within the received real-time images, articulate the distal end of the EWC using the drive mechanism to align the distal end of the EWC with the identified centerpoint, and instruct the drive mechanism to reduce tension on the distal end of the EWC to permit the distal end of the EWC to deflect when contacting walls of the lumen if a diameter of the lumen approximates an outer diameter of the EWC.

### BACKGROUND

### Technical Field

The present disclosure relates to the field of navigating medical devices within a patient, and in particular, planning a pathway though a luminal network of a patient and navigating medical devices to a target.

### Description of Related Art

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), cone-beam computed tomography (CBCT) or fluoroscopy (including 3D fluoroscopy) are employed by clinicians to identify and navigate to areas of interest within a patient and ultimately a target for biopsy or treatment. In some procedures, pre-operative scans may be utilized for target identification and intraoperative guidance. However, real-time imaging may be required to obtain a more accurate and current image of the target area. Furthermore, real-time image data displaying the current location of a medical device with respect to the target and its surroundings may be needed to navigate the medical device to the target in a safe and accurate manner (e.g., without causing damage to other organs or tissue).

For example, an endoscopic approach has proven useful in navigating to areas of interest within a patient. To enable the endoscopic approach endoscopic navigation systems have been developed that use previously acquired MRI data or CT image data to generate a three-dimensional (3D) rendering, model, or volume of the particular body part such as the lungs.

The resulting volume generated from the MRI scan or CT scan is then utilized to create a navigation plan to facilitate the advancement of the endoscope (or other suitable medical device) within the patient anatomy to an area of interest. A locating or tracking system, such as an electromagnetic (EM) tracking system, may be utilized in conjunction with, for example, CT data, to facilitate guidance of the endoscope to the area of interest.

However, manually navigating the endoscope through the luminal network or utilizing robotic controls to remotely navigate the endoscope through the luminal network can be complex and time consuming. Further, navigating the endoscope through the luminal network takes considerable skill to ensure no damage is done to the surrounding tissue and that the endoscope is navigated to the correct location.

### SUMMARY

A system for performing a surgical procedure includes an extended working channel (EWC), the EWC including a drive mechanism configured to articulate a distal end of the EWC, a catheter operably coupled to the EWC, the catheter including a camera and an electromagnetic (EM) sensor, and a workstation operably coupled to the EWC and the catheter, the workstation including a memory and a processor, the memory storing instructions, which when executed by the processor cause the processor to determine a location and an orientation of the distal end of the EWC, receive real-time images of the patient's anatomy from the camera of the catheter, identify a centerpoint of a lumen within the received real-time images of the patient's anatomy, determine if a center of the catheter is aligned with the identified centerpoint of the lumen, articulate, if the catheter is misaligned with the identified centerpoint of the lumen, the distal end of the EWC using the drive mechanism to align the distal end of the EWC with the identified centerpoint of the lumen, determine if the distal end of the EWC is located within an airway having a diameter that approximates an outer diameter of the catheter, and instruct, if the distal end of the EWC is located within an airway having a diameter that approximates the outer diameter of the catheter, the drive mechanism to reduce tension on the distal end of the EWC to permit the distal end of the EWC to deflect when contacting walls of the lumen.

In aspects, the memory may store thereon further instructions, which when executed by the processor cause the processor to generate a three-dimension (3D) representation of a scene distal of the camera of the catheter.

In other aspects, the memory may store thereon further instructions, which when executed by the processor cause the processor to generate the 3D representation of the scene using pixel-wise depth estimation.

In certain aspects, the memory may store thereon further instructions, which when executed by the processor cause the processor to identify the centerpoint of the lumen using data resulting from the pixel-wise depth estimation.

In other aspects, the memory may store thereon further instructions, which when executed by the processor cause the processor to generate a 3D model of the airways of the patient using pre-procedure images of the patient's anatomy.

In aspects, the memory may store thereon further instructions, which when executed by the processor cause the processor to generate a pathway through the airways of the patient to target tissue identified in the generated 3D model.

In certain aspects, the memory may store thereon further instructions, which when executed by the processor cause the processor to identify a bifurcation within the received real-time images of the patient's anatomy.

In other aspects, the memory may store thereon further instructions, which when executed by the processor cause the processor to articulate the distal end of the EWC using the drive mechanism to align the distal end of the EWC with a lumen of the bifurcation associated with the pathway of the target tissue.

In aspects, the memory may store thereon further instructions, which when executed by the processor cause the processor to determine if a position of the distal end of the EWC within the patient's airways has changed within a predetermined time period.

In certain aspects, the memory may store thereon further instructions, which when executed by the processor cause the processor to disable the drive mechanism if it is determined that the position of the distal end of the EWC has not changed within the predetermined time period.

In accordance with another aspect of the disclosure, a method for navigating a surgical device to an area of interest includes determining a pose of a distal end of an extended working channel using an inertial measurement unit coupled to the EWC, receiving real-time images of a patient's anatomy from a camera coupled to a catheter, the catheter received within a portion of the EWC, identifying a centerpoint of a lumen within the received real-time images of the patient's anatomy, determining if a center of the catheter is aligned with the identified centerpoint of the lumen, articulating, if the catheter is misaligned with the identified centerpoint of the lumen, the distal end of the EWC using a drive mechanism to align the distal end of the EWC with the identified centerpoint of the lumen, wherein the drive mechanism is operably coupled to the EWC, determining if the distal end of the EWC is located within an airway having a diameter that approximates an outer diameter of the catheter, and instructing, if the distal end of the EWC is located within an airway having a diameter that approximates the outer diameter of the catheter, the drive mechanism to reduce tension on the distal end of the EWC to permit the distal end of the EWC to deflect when contacting walls of the lumen.

In aspects, the method may include generating a three-dimensional (3D) representation of a scene distal of the camera of the catheter.

In other aspects, the 3D representation of the scene may be generated using pixel-wise depth estimation.

In certain aspects, the method may include determining if a position of the distal end of the EWC within the patient's airways has changed within a predetermined time period.

In other aspects, the method may include disabling the drive mechanism if it is determined that the position of the distal end of the EWC has not changed within the predetermined time period.

In accordance with another aspect of the disclosure, a method of navigating a surgical device to an area of interest includes determining a pose of a distal end of an extended working channel (EWC) using an inertial measurement unit coupled to the EWC, receiving real-time images of a patient's anatomy from a camera coupled to a catheter, wherein the catheter is received within a portion of the EWC, articulating the distal end of the EWC, using a drive mechanism coupled to the EWC, to align a distal portion of the catheter with a lumen of a bifurcation identified within the received real-time images, and instructing the drive mechanism to reduce tension on the distal end of the EWC if a determined outer dimension of the lumen approximates an outer dimension of the catheter to permit the distal end of the EWC to deflect when contacting walls of the lumen.

In aspects, the method may include generating a three-dimensional (3D) representation of a scene distal of the camera of the catheter.

In certain aspects, the 3D representation of the scene may be generated using pixel-wise depth estimation.

In other aspects, the method may include determining if a position of the distal end of the EWC within the patient's airways has changed within a predetermined time period.

In aspects, the method may include disabling the drive mechanism if it is determined that the position of the distal end of the EWC has not changed within the predetermined time period.

### BRIEF DESCRIPTION OF THE DRAWINGS

Various aspects and embodiments of the disclosure are described hereinbelow with references to the drawings, wherein:

FIG. 1 is a schematic view of a surgical system provided in accordance with the present disclosure;

FIG. 2 is an exploded view of a drive mechanism of an extended working channel of the surgical system of FIG. 1;

FIG. 3 is a perspective view of a distal portion of a catheter of the surgical system of FIG. 1;

FIG. 4 is a schematic view of a workstation of the surgical system of FIG. 1;

FIG. 5 is a depiction of a graphical user interface of the surgical system of FIG. 1 illustrating a 3D representation of a patient's airways and a pathway to target tissue;

FIG. 6 is a depiction of the graphical user interface of FIG. 5 illustrating a scene of the patient's airways distal of the catheter of FIG. 3 and an identified center of the airway;

FIG. 7 is a depiction of the graphical user interface of FIG. 5 illustrating a scene of the patient's airways distal of the catheter of FIG. 3 and an identified bifurcation of the airways;

FIG. 8A is a flow diagram of a method of performing a surgical procedure in accordance with the disclosure;

FIG. 8B is a continuation of the flow diagram of FIG. 8A;

FIG. 8C is a continuation of the flow diagram of FIG. 8C; and

FIG. 9 is a perspective view of a robotic surgical system of the surgical system of FIG. 1.

### DETAILED DESCRIPTION

The present disclosure is directed to a surgical system configured to enable navigation of a medical device through a luminal network of a patient, such as for example the lungs. The surgical system includes a bronchoscope, through which a first catheter, such as for example, an extended working channel (EWC), which may be a smart extended working channel (sEWC), is advanced to permit access of a second catheter to the luminal network of the patient. The sEWC includes an electromagnetic (EM) sensor disposed on or adjacent to a distal end of the sEWC that is configured for use with an electromagnetic navigation (EMN) or tracking system, which tacks the location of EM sensors, such as for example, the EM sensor of the sEWC. The second catheter includes a camera disposed on or adjacent to a distal end of the second catheter that is configured to capture real-time images of the patient's anatomy as the second catheter is navigated through the luminal network of the patient. In this manner, the second catheter is advanced through the sEWC and into the luminal network of the patient. It is envisioned that the second catheter may be selectively locked to the sEWC to selectively inhibit, or permit, movement of the second catheter relative to the sEWC. In embodiments, the second catheter may include an EM sensor disposed on or adjacent to the distal end of the second catheter. It is envisioned that the second catheter may include an inertial measurement unit to aid in determining a pose of the second catheter within the airways of the patient. The sEWC includes a drive mechanism that is configured to articulate a distal end of the sEWC. In this manner, the sEWC may be manually advanced, retracted, or rolled within the patient's airways and the drive mechanism automatically articulates the distal end of the sEWC to maintain alignment of a center portion of the second catheter with a center point of the lumen distal of the camera and to follow the pathway to the target tissue. In embodiments, the drive mechanism may articulate the distal end of the sEWC to be disposed off-center of the center of the lumen (e.g., for example, adjacent a sidewall of the lumen) in situations where the sEWC encounters a sharp corner necessitating acute bends of the SEWC.

The surgical system generates a 3-dimensional (3D) representation of the airways of the patient using pre-procedure images, such as for example, CT, CBCT, or MRI images and enables the identification of target tissue within the 3D representation and the generation of a pathway through the patient's airways to the target tissue. During navigation of the sEWC and second catheter within the airways of the patient, the camera captures real-time images of the patient's anatomy distal of the camera. The captured real-time images are analyzed and a 3D representation of a scene of the patient's anatomy distal of the camera is generated using pixel-wise depth estimation, although it is contemplated that any suitable method may be utilized to generate the 3D representation of the scene without departing from the scope of the disclosure. Although generally described as utilizing a camera, it is envisioned that the second catheter may include any suitable imaging modality, such as for example, ultrasound and infrared (IR) sensors, and combinations thereof. A centerpoint of the lumen within the 3D representation of the scene is determined and any bifurcations within the 3D representation of the scene are identified. The system automatically aligns the center portion of the second catheter with the centerpoint of the lumen and if any bifurcations are present, aligns the catheter with a lumen of the bifurcation associated with the pathway to the target tissue. As can be appreciated, the automatic articulation of the distal end of the sEWC is effectuated contemporaneously with advancement of the sEWC and second catheter within the airways of the patient.

The system identifies a diameter of the lumen(s) identified in the real-time images and compares the identified diameter to an outer dimension of the second catheter. As can be appreciated, as the lumens become smaller and smaller, it becomes increasingly more difficult to identify a centerpoint of the lumen and therefore, the likelihood of damaging the airways increases. To mitigate potential damage to the walls of the airways, if the diameter of the lumen approximates the outer dimension of the second catheter the system adjusts the tension applied to the pull wires of the sEWC to permit the sEWC to be deflected when the second catheter abuts or otherwise contacts the walls of the lumen. In this manner, the system reduces the tension applied to the pull wires of the sEWC, although it is contemplated that the system may increase the tension applied to the pull wires of the sEWC depending upon the needs of the procedure being performed.

In embodiments, the system monitors an amount of time the sEWC remains stationary within the airways, and if the sEWC remains stationary for longer than a predetermine time period, the system disables the drive mechanism to inhibit automatic articulation of the distal end of the sEWC and permit manual articulation of the distal end of the sEWC. These and other aspects of the disclosure will be described in further detail hereinbelow. Although generally described with reference to the lung, it is contemplated that the systems and methods described herein may be used with any structure within the patient's body, such as for example, the liver, the kidney, the prostate, the heart, the brain, and gynecological.

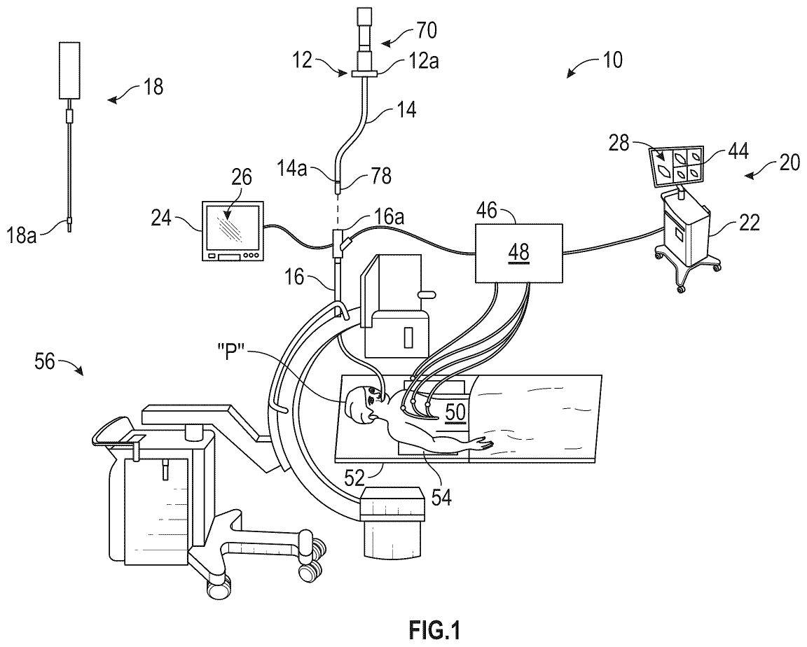

Turning now to the drawings, FIG. 1 illustrates a system 10 in accordance with the disclosure facilitating navigation of a medical device through a luminal network and to an area of interest. The system 10 includes a catheter guide assembly 12 including a first catheter, which in embodiments may be an extended working channel (EWC) 14, which may be a smart extended working channel (sEWC) including an electromagnetic (EM) sensor. It is contemplated that the first catheter may be any suitable catheter capable of being navigated within the patient's luminal network, and in one non-limiting embodiment, may be a continuum manipulator. In one embodiment, the sEWC 14 is inserted into a bronchoscope 16 for access to a luminal network of the patient P. In this manner, the sEWC 14 may be inserted into a working channel of the bronchoscope 16 for navigation through a patient's luminal network, such as for example, the lungs. It is envisioned that the sEWC 14 may itself include imaging capabilities via an integrated camera or optics component (not shown) and therefore, a separate bronchoscope 16 is not strictly required. In embodiments, the sEWC 14 may be selectively locked to the bronchoscope 16 using a bronchoscope adapter 16a. In this manner, the bronchoscope adapter 16a is configured to permit motion of the sEWC 14 relative to the bronchoscope 16 (which may be referred to as an unlocked state of the bronchoscope adapter 16a) or inhibit motion of the sEWC 14 relative to the bronchoscope 16 (which may be referred to as a locked state of the bronchoscope adapter 16a). Bronchoscope adapters 16a are currently marketed and sold by Medtronic PLC under the brand names EDGE® Bronchoscope Adapter or the ILLUMISITE® Bronchoscope Adapter and are contemplated as being usable with the disclosure.

As compared to an EWC, the sEWC 14 includes one or more EM sensors 14a disposed in or on the sEWC 14 at a predetermined distance from the distal end of the sEWC 14. As can be appreciated, the EM sensor 14a is a separate EM sensor from other EM sensors of the system 10 (such as for example, a locatable guide (LG), a catheter, a bronchoscope). It is contemplated that the EM sensor 14a may be a five degree-of-freedom sensor or a six-degree-of-freedom sensor. It is envisioned that the sEWC 14. It is contemplated that the sEWC 14 may be utilized together with an LG, a catheter, or the bronchoscope 16, in which data from the EM sensors 14a of the sEWC 14, the catheter, and/or the bronchoscope 16 may be fused together. As can be appreciated, the position and orientation of the EM sensor 14a to a reference coordinate system, and thus a distal portion of the sEWC 14, within an electromagnetic field can be derived. Catheter guide assemblies 12 are currently marketed and sold by Medtronic PLC under the brand names SUPERDIMENSION® Procedure Kits, ILLUMISITE™ Endobronchial Procedure Kit, ILLUMISITE™ Navigation Catheters, or EDGE® Procedure Kits, and are contemplated as being usable with the disclosure.

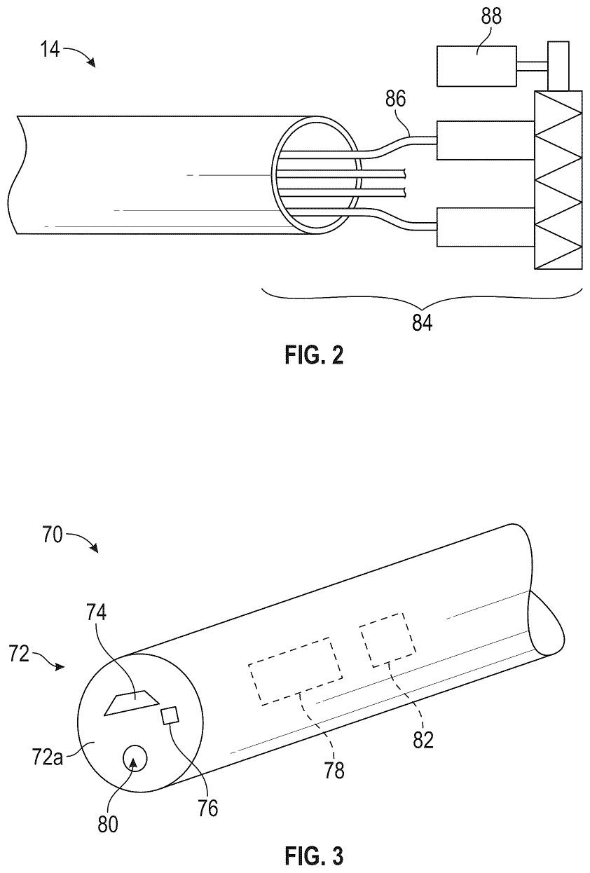

With additional reference to FIG. 2, articulation of the distal end of the sEWC 14 is effectuated electronically via a drive mechanism 84 disposed within an interior portion of the sEWC 14. The drive mechanism 84 effectuates manipulation or articulation of the distal end of the sEWC 14 in four degrees of freedom or two planes of articulation (e.g., for example, left, right, up, down), which is controlled by two push-pull wires 86, although it is contemplated that the drive mechanism 84 may include any suitable number of wires to effectuate movement or articulation of the distal end of the sEWC 14 in greater or fewer degrees of freedom without departing form the scope of the disclosure. It is contemplated that the distal end of the sEWC 14 may be manipulated in more than two planes of articulation, such as for example, in polar coordinates, or may maintain an angle of the distal end relative to the longitudinal axis of the sEWC 14 while altering the azimuth of the distal end of the sEWC 14 or vice versa. In one non-limiting embodiment, the system 10 may define a vector or trajectory of the distal end of the sEWC 14 in relation to the two planes of articulation.

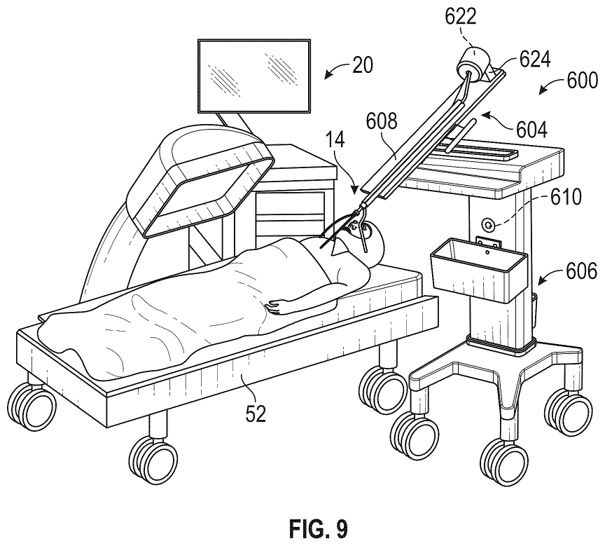

It is envisioned that the drive mechanism 84 may be cable actuated using artificial tendons or pull wires 86 (e.g., for example, metallic, non-metallic, and/or composite) or may be a nitinol wire mechanism. In embodiments, the drive mechanism 84 may include motors 88 or other suitable devices capable of effectuating movement of the pull wires 86. In this manner, the motors 88 are disposed within the sEWC 14 such that rotation of an output shaft the motors 88 effectuates a corresponding articulation of the distal end of the sEWC 14. Although generally described as having the motors 88 disposed within the sEWC 14, it is contemplated that the sEWC 14 may not include motors 88 disposed therein. Rather, the drive mechanism 84 disposed within the sEWC 14 may interface with motors disposed within a portion of a robotic surgical system (FIG. 9), as will be described in further detail hereinbelow. As can be appreciated, by removing the motors 88 from the sEWC 14, the sEWC 14 becomes increasingly cheaper to manufacture and may be a disposable unit. As will be described in further detail hereinbelow, in one-non-limiting embodiment, advancement and retraction of the sEWC 14 within the luminal network of the patient P is manually controlled while articulation of the distal end of the sEWC is effectuated by the system 10 and the drive mechanism 84.

Returning to FIG. 1 and with additional reference to FIG. 3, the system 10 includes a second second catheter 70 configured to be inserted into, and extend from the sEWC 14. The second second catheter 70 includes a distal portion 72 defining a distal surface 72a. In embodiments, the second second catheter 70 may be selectively locked into position relative to the sEWC 14 such that at least the distal surface 72a extends beyond the distal tip of the sEWC 14. It is contemplated that the second catheter 70 may be selectively locked into position relative to the sEWC 14 such that one or more EM sensors 78 disposed on or within the distal portion 72 of the second catheter 70 extends a predetermined distance beyond the distal tip of the sEWC 14. In this manner, the system 10 is able to determine a position of a distal portion of the second catheter 70 within the luminal network of the patient P. As can be appreciated, the EM sensor 78 disposed on the second catheter 70 is separate from the EM sensor 14a disposed on the sEWC 14. It is envisioned that the second catheter 70 may be selectively locked relative to the sEWC 14 at any time, regardless of the position of the distal portion 76 of the second catheter 70 relative to the sEWC 14. In embodiments, the second catheter 70 may be selectively locked to a handle 12a of the catheter guide assembly 12 using any suitable means, such as for example, a snap fit, a press fit, a friction fit, a cam, one or more detents, threadable engagement, or a chuck clamp. It is envisioned that the EM sensor 78 may be a five degree-of-freedom sensor or a six degree-of-freedom sensor. As will be described in further detail hereinbelow, the position and orientation of the EM sensor 78 of the second catheter 70 relative to a reference coordinate system, and thus a distal portion of the second catheter 70, within an electromagnetic field can be derived.

At least one camera 74 is disposed on or adjacent to the distal surface 72a of the second catheter 70 and is configured to capture, for example, still images, real-time images, or real-time video. Although generally described as being disposed on the distal surface 72a of the second catheter 70, it is envisioned that the camera 74 may be disposed on any suitable location on the second catheter 70, such as for example, a sidewall. In embodiments, the second catheter 70 may include one or more light sources 76 disposed on or adjacent to the distal surface 72a or any other suitable location (e.g., for example, a side surface or a protuberance). The light source 76 may be or may include, for example, a light emitting diode (LED), an optical fiber connected to a light source that is located external to the patient P, or combinations thereof, and may emit one or more of white, infrared (IR), or near infrared (NIR) light (such as for example, Near-Infrared Fluorescence). In this manner, the camera 74 may be, for example, a white light camera, an IR camera, or an NIR camera that is capable of capturing white light and NIR light, or combinations thereof. In one non-limiting embodiment, the camera 74 is a white light mini complementary metal-oxide semiconductor (CMOS) camera, although it is contemplated that the camera 74 may be any suitable camera, such as for example, a charge-coupled device (CCD), a N-type metal-oxide-semiconductor (NMOS), and in embodiments, may be a dual lens camera, a Red Blue Green and Depth (RGB-D) camera, a white light stereo camera, or other suitable camera configured to identify a distance between the camera 74 and anatomical features within the patient's anatomy without departing from the scope of the disclosure. In embodiments, the camera 74 may be a camera configured to differentiate a contrast agent from surrounding tissue or may be an ultrasound probe. Although generally described as utilizing the camera 74, it is envisioned that the second catheter 70 may be an ultrasound catheter and in embodiments, it is envisioned that the system 10 may utilize computed tomography (CT), cone-beam computed tomography (CBCT), fluoroscopic imaging, or other suitable external imaging modalities without departing from the scope of the disclosure.

Continuing with FIG. 3, in embodiments, the second catheter 70 may include a working channel 80 defined through a proximal portion (not shown) and the distal surface 72a, although in embodiments, it is contemplated that the working channel 80 may extend through a sidewall of the second catheter 70 depending upon the design needs of the second catheter 70. The second catheter 70 includes an inertial measurement unit (IMU) 82 disposed within or adjacent to the distal portion 72. As can be appreciated, the IMU 82 detects an orientation of the distal portion 72 of the second catheter 70 relative to a reference coordinate frame and detects movement and speed of the distal portion 72 of the second catheter 70 as the second catheter 70 is navigated within the patient's luminal network. Using the data received from the IMU 82, the system 10 is able to determine alignment and trajectory information of the distal portion 72 of the second catheter 70. In embodiments, the system 10 may utilize the data received from the IMU 82 to determine a gravity vector, which may be used to determine the orientation of the distal portion 72 of the second catheter 70 within the airways of the patient P. Although generally described as using the IMU 82 to detect an orientation and/or movement of the distal portion 72 of the second catheter 70, the instant disclosure is not so limited and may be used in conjunction with flexible sensors, such as for example, fiber-bragg grating sensors, ultrasonic sensors, without sensors, or combinations thereof. As will be described in further detail hereinbelow, it is contemplated that the devices and systems described herein may be used in conjunction with robotic systems such that robotic actuators drive the sEWC 14 or bronchoscope 16 proximate the target.

Returning again to FIG. 1, it is envisioned that a locatable guide (LG) 18, including one or more EM sensors 18a may be inserted into the sEWC 14 and selectively locked into position relative to the sEWC 14 such that the sensor 18a extends a desired distance beyond a distal tip of the sEWC 14. As can be appreciated, the sensor 18a is disposed on or in the LG 18 a predetermined distance from a distal end of the LG 18. It is contemplated that the EM sensor 18a may be a five degree-of-freedom sensor or a six degree-of-freedom sensor. In embodiments, the LG 18 may be locked relative to the sEWC 14 such that the EM sensor 18a of the LG 18 extends a first, predetermined fixed distance beyond the distal tip of the sEWC 14 to enable the system 10 to determine a position of a distal portion of the LG 18 within the luminal network of the patient. It is envisioned that the LG 18 may be selectively locked relative to the sEWC 14 at any time, regardless of the position of the distal end of the LG 18 relative to the sEWC 14. It is contemplated that the LG 18 may be selectively locked to a handle 12a of the catheter guide assembly 12 using any suitable means, such as for example, a snap fit, a press fit, a friction fit, a cam, one or more detents, threadably engagement, or a chuck clamp.

With continued reference to FIG. 1, the system 10 generally includes an operating table 52 configured to support the patient P and monitoring equipment 24 coupled to the sEWC 14, the bronchoscope 16, the second catheter 70, or combinations thereof (e.g., for example, a video display for displaying the video images received from the video imaging system of the bronchoscope 12 or the camera 74 of the second catheter 70), a locating or tracking system 46 including a tracking module 48, a plurality of reference sensors 50 and a transmitter mat 54 including a plurality of incorporated markers, and a workstation 20 having a computing device 22 including software and/or hardware used to facilitate identification of a target, pathway planning to the target, navigation of a medical device to the target, and/or confirmation and/or determination of placement of, for example, the sEWC 14, the bronchoscope 16, the second catheter 70, or a surgical tool, relative to the target.

The tracking system 46 is, for example, a six degrees-of-freedom electromagnetic locating or tracking system, or other suitable system for determining a position and orientation of, for example, a distal portion of the sEWC 14, the bronchoscope 16, the second catheter 70, or a surgical tool, for performing registration of a detected position of one or more of the EM sensors 14a or 78 and a three-dimensional (3D) model generated from a CT, CBCT, or MRI image scan. The tracking system 46 is configured for use with the sEWC 14 and the second catheter 70, and particularly with the EM sensors 14a and 78.

Continuing with FIG. 1, the transmitter mat 54 is positioned beneath the patient P. The transmitter mat 54 generates an electromagnetic field around at least a portion of the patient P within which the position of the plurality of reference sensors 50 and the EM sensors 14a and 78 can be determined with the use of the tracking module 48. In one non-limiting embodiment, the transmitter mat 54 generated three or more electromagnetic fields. One or more of the reference sensors 50 are attached to the chest of the patient P. In embodiments, coordinates of the reference sensors 50 within the electromagnetic field generated by the transmitter mat 54 are sent to the computing device 22 where they are used to calculate a patient coordinate frame of reference (e.g., for example, a reference coordinate frame). As will be described in further detail hereinbelow, registration is generally performed using coordinate locations of the 3D model and 2D images from the planning phase, with the patient P's airways as observed through the bronchoscope 12 or second catheter 70 and allow for the navigation phase to be undertaken with knowledge of the location of the EM sensors 14a and 78. It is envisioned that any one of the EM sensors 14a and 78 may be a single coil sensor that enables the system 10 to identify the position of the sEWC or the second catheter 70 within the EM field generated by the transmitter mat 54, although it is contemplated that the EM sensors 14a and 78 may be any suitable sensor and may be a sensor capable of enabling the system 10 to identify the position, orientation, and/or pose of the sEWC 14 or the second catheter 70 within the EM field. Although generally described with respect to EMN systems using EM sensors, the instant disclosure is not so limited and may be used in conjunction with flexible sensors, such as for example, fiber bragg grating sensors, IMU's (such as for example, the IMU 82 or additional IMU's), ultrasonic sensors, optical sensors, pose sensors (e.g., for example, ultra-wide band, global positioning system, fiber-bragg, radio-opaque markers), with out sensors or combinations thereof.

In accordance with aspects of the disclosure, the visualization of intra-body navigation of a medical device (e.g., for example, a biopsy tool or a therapy tool), towards a target (e.g., for example, a lesion) may be a portion of a larger workflow of a navigation system. An imaging device 56 (e.g., for example, a CT imaging device, such as for example, a CBCT device, including but not limited to Medtronic plc's O-arm™ system) capable of acquiring 2D and 3D images or video of the patient P is also included in the particular aspect of system 10. The images, sequence of images, or video captured by the imaging device 56 may be stored within the imaging device 56 or transmitted to the computing device 22 for storage, processing, and display. In embodiments, the imaging device 56 may move relative to the patient P to create a sequence of images, such as for example, a fluoroscopic video. The pose of the imaging device 56 relative to the patient P while capturing the images may be estimated via markers incorporated with the transmitter mat 54. The markers are positioned under the patient P, between the patient P and the operating table 52, and between the patient P and a radiation source or a sensing unit of the imaging device 56. The markers incorporated with the transmitter mat 54 may be two separate elements which may be coupled in a fixed manner or alternatively may be manufactured as a single unit. It is contemplated that the imaging device 56 may include a single imaging device or more than one imaging device.

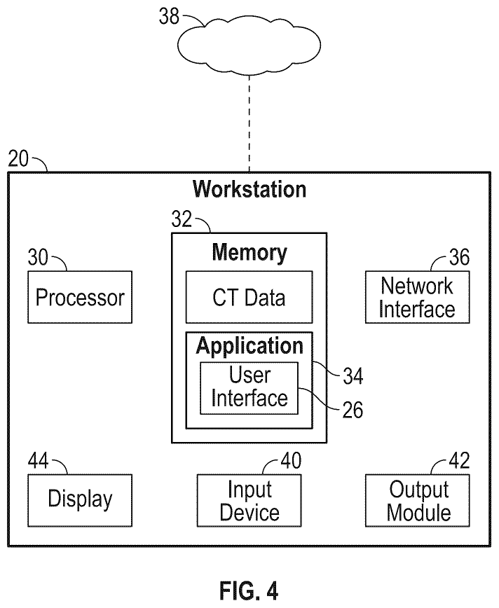

Continuing with FIG. 1, and with additional reference to FIG. 4, the workstation 20 includes a computer 22 and a display 24 that is configured to display one or more user interfaces 26 and/or 28. The workstation 20 may be a desktop computer or a tower configuration with the display 24 or may be a laptop computer or other computing device. The workstation 20 includes a processor 30 which executes software stored in a memory 32. The memory 32 may store video or other imaging data captured by the bronchoscope 16 or the second catheter 70 or pre-procedure images from, for example, a CT scan, Positron Emission Tomography (PET), Magnetic Resonance Imaging (MRI), or CBCT. In addition, the memory 32 may store one or more software applications 34 to be executed on the processor 30. Though not explicitly illustrated, the display 24 may be incorporated into a head mounted display such as an augmented reality (AR) headset such as the HoloLens offered by Microsoft Corp.

A network interface 36 enables the workstation 20 to communicate with a variety of other devices and systems via the Internet. The network interface 36 may connect the workstation 20 to the Internet via a wired or wireless connection. Additionally, or alternatively, the communication may be via an ad-hoc Bluetooth® or wireless network enabling communication with a wide-area network (WAN) and/or a local area network (LAN). The network interface 36 may connect to the Internet via one or more gateways, routers, and network address translation (NAT) devices. The network interface 36 may communicate with a cloud storage system 38, in which further image data and videos may be stored. The cloud storage system 38 may be remote from or on the premises of the hospital such as in a control or hospital information technology room. An input module 40 receives inputs from an input device such as, for example, a keyboard, a mouse, or voice commands. An output module 42 connects the processor 30 and the memory 32 to a variety of output devices such as, for example, the display 24. In embodiments, the workstation 20 may include its own display 44, which may be a touchscreen display.

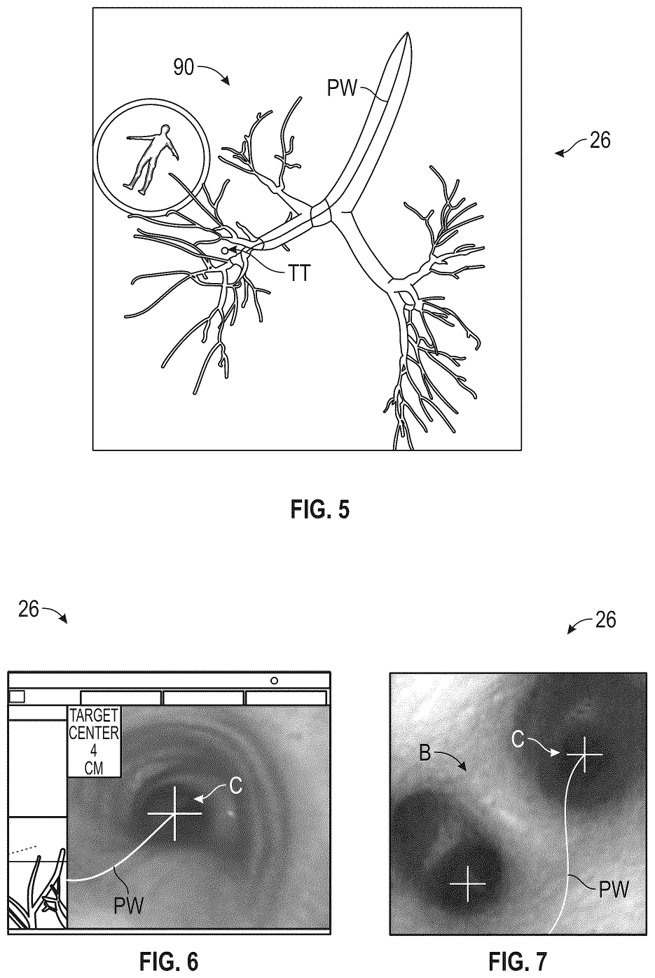

In a planning or pre-procedure phase, the software application utilizes pre-procedure CT image data, ether stored in the memory 32 or retrieved via the network interface 36, for generating and viewing a 3D model 90 (FIG. 5) of the patient P's anatomy, enabling the identification of target tissue TT on the 3D model 90 (automatically, semi-automatically, or manually), and in embodiments, allowing for the selection of a pathway through the patient P's anatomy to the target tissue. Examples of such an application is the ILOGIC® planning and navigation suites and the ILLUMISITE® planning and navigation suites currently marketed by Medtronic PLC. The 3D model 90 may be displayed on the display 24 or any other suitable display associated with the workstation 20, various views of the 3D model 90 may be provided and/or the 3D model may be manipulated to facilitate identification of target tissue on the 3D model 90 and/or selection of a suitable pathway to the target tissue.

In embodiments, the software stored in the memory 32 may identify and segment out a targeted critical structure within the 3D model 90. It is envisioned that the segmentation process may be performed automatically, manually, or a combination of both. The segmentation process isolates the targeted critical structure from the surrounding tissue in the 3D model 90 and identifies its position within the 3D model 90. As can be appreciated, this position can be updated depending upon the view selected on the display 24 such that the view of the segmented targeted critical structure may approximate a view captured by the camera 74 of the second catheter 70.

Registration of the patient P's location on the transmitter mat 54 may be performed by moving the EM sensors 14a, 18a, or 78 through the airways of the patient P. In this manner, data pertaining to the locations of the EM sensors 14a, 18a, or 78, while the sEWC 14, the LG 18, or the second catheter 70 is moving through the airways, is recorded using the transmitter mat 54, the reference sensors 50, and the tracking system 46. A shape resulting from this location data is compared to an interior geometry of passages of the 3D model 90, and a location correlation between the shape and the 3D model based on the comparison is determined, e.g., for example, utilizing the software on the computing device 22. In addition, the software identifies non-tissue space (e.g., for example, air filled cavities) in the 3D model 90. The software aligns, or registers, an image representing a location of the EM sensors 14a, 18a, or 78 with the 3D model 90 and/or 2D images generated from the 3D model 90, which are based on the recorded location data and an assumption that the sEWC 14, the LG 18, or the second catheter 70 remains located in non-tissue space in a patient P's airways. In embodiments, a manual registration technique may be employed by navigating the sEWC 14, the LG 18, or the second catheter 70 with the EM sensors 14a, 18a, or 78 to pre-specified locations in the lungs of the patient P, and manually correlating the images from the bronchoscope 16 or the second catheter 70 to the model data of the 3D model 90.

With additional reference to FIGS. 5-7, during a navigation phase, the sEWC 14, along with the second catheter 70, is manually advanced, retracted, or rolled within the luminal network of the patient P and the distal end of the sEWC 14 is automatically articulated and/or manipulated via the software stored in the memory 32 and the drive mechanism 84. In this manner, utilizing the selected pathway to the target tissue in conjunction with pre-procedure images stored in the memory 32 and/or real-time images captured by the camera 74 of the second catheter 70, the software stored in the memory 32 identifies the location of the distal portion 72 of the second catheter 70 within the patient P's luminal network and/or relative to the selected pathway “PW” to the target tissue “TT” and identifies a position of the distal portion 72 of the second catheter 70 relative to a center “C” or centroid of the lumen of the patient P's luminal network in which the sEWC 14 and second catheter 70 are disposed. In this manner, as the sEWC 14 and the second catheter 70 are advanced within the luminal network of the patient P, the images captured by the camera 74 of the second catheter 70 are processed by the software stored in the memory 32 to generate a 3D representation of the patient P's anatomy within the field of view (FOV) of the camera 74 (e.g., for example, a 3D representation of the scene in front of the second catheter 70).

It is envisioned that the software stored in the memory 32 may process the real-time images captured by the camera 74 using pixel-wise depth estimation to represent the scene in front of the second catheter 70 as a point cloud. Using the generated point cloud, the software stored in the memory 32 identifies the center C of the lumen within the scene and the position of the distal portion 72 of the second catheter 70 relative to the identified center C of the lumen. Using data collected by the IMU 82 in conjunction with, or separate from the position data obtained by the EM sensors 14a and/or 82, a pose of the distal portion 72 of the second catheter 70 relative to the reference coordinate frame is determined. With pose of the distal portion 72 of the second catheter 70 and the location of the distal portion 72 of the second catheter 70 relative to the identified center C of the lumen determined, the software stored in the memory 32 instructs the drive mechanism 84 to articulate the distal end of the sEWC 14 in a direction towards the identified center C of the lumen to aim or otherwise align the distal portion 72 of the second catheter 70 with the identified center C of the lumen. As can be appreciated, the software stored in the memory 32 continuously processes the images captured by the camera 74 in real-time to ensure the distal portion 72 of the second catheter 70 is aligned with the center C of the lumen to inhibit or otherwise minimize the chances of the distal portion 72 of the second catheter 70 from contacting a sidewall of the lumen.

In embodiments, using the pose of the distal portion 72 or the second catheter 70 and the location of the distal portion 72 of the second catheter 70 relative to the identified center of the lumen, the software stored in the memory 32 may identify a radius of curvature of the lumen or bifurcation along the center of the selected pathway PW and compare the identified radius of curvature to a minimum bend radius of the sEWC 14. If the identified radius of curvature of the lumen is below a minimum threshold value (e.g., for example, the minimum bend radius of the sEWC 14), the software stored in the memory 32 instructs the drive mechanism 84 to articulate the distal end of the sEWC 14 to a location within the lumen where the radius of curvature of the lumen is greater than or equal to the minimum threshold value. As can be appreciated, this location may be off-center or adjacent a wall of the lumen to maximize the radius of curvature available within the confines of the lumen.

When the distal portion 72 of the second catheter 70 encounters a bifurcation B within the patient P's luminal network, the software stored in the memory 32 identifies the lumen through which the sEWC 14 and second catheter 70 must travel to remain on the selected pathway PW to the target tissue TT. At this point, the software stored in the memory 32 instructs the drive mechanism 84 to manipulate the distal end of the sEWC 14 to align the distal end portion 72 of the second catheter 70 with the identified center C of the appropriate lumen and continued manual advancement of the sEWC 14 and second catheter 70 within the patient P's luminal network is performed.

In embodiments, the software stored in the memory 32 detects a lack of manual manipulation (e.g., for example, advancement, retraction, or roll) of the sEWC 14 and second catheter 70 and may enable manual control of articulation of the distal end of the sEWC 14 (e.g., for example, overriding automatic articulation of the distal end of the sEWC 14). It is envisioned that the software stored in the memory 32 may identify a lack of manual manipulation of the sEWC 14 by comparing a determined length of time since the sEWC 14 was manipulated to a predetermined threshold. In this manner, if the determined length of time is less than the predetermined threshold, automatic articulation by the software stored in the memory 32 is maintained and if the determined length of time is greater than the predetermined threshold, automatic articulation by the software stored in the memory 32 is disabled and manual control of articulating the distal end of the sEWC 14 is enabled. As can be appreciated, the software stored in the memory 32 can continuously evaluate the length of time since the sEWC 14 was manually manipulated and enable and disable automatic control of articulation of the distal end of the sEWC 14 as many times as is necessary.

As the distal portion 72 of the second catheter 70 is further advanced within the luminal network of the patient P, the software stored in the memory 32 identifies whether the distal portion 72 of the catheter has reached a narrow airway, at which point if manual manipulation of the sEWC 14 is detected within the predetermined threshold, the software stored in the memory 32 instructs the drive mechanism 84 to adjust the tension applied to the pull wires 86, and therefore, the distal end of the sEWC 14. In this manner, the software stored in the memory 32 may increase or decrease the tension applied to the pull wires 86 depending upon the needs of the procedure being performed. As can be appreciated, the software stored in the memory 32 may not be able to accurately identify a center point C of a narrow airway or may not be able to accurately articulate the distal end of the sEWC 14 to maintain alignment of the distal end of the sEWC 14 with the center of the narrow airway. Therefore, the likelihood that the distal end of the sEWC 14 or the distal portion 72 of the second catheter 70 may contact a wall of the narrow airway is greater. Reducing the tension applied to the pull wires 86 of the sEWC 14 permits the distal end of the sEWC 14 to be deflected when contacting walls of the narrow airway and minimize injury to the patient P's airways caused by the sEWC 14 and/or second catheter 70. It is envisioned that when encountering a bifurcation B within the narrow airways of the patient P's lungs, the software stored in the memory 32 may automatically manipulate the distal end of the sEWC 14 to substantially align the distal end of the sEWC 14 with the appropriate lumen and then relax the tension on the pull wires 86 of the sEWC during further manual advancement of the sEWC 14 and second catheter 70 within the appropriate lumen to enable deflection of the distal end of the sEWC 14 by the airway walls.

Although generally described as utilizing pixel-wise depth estimation, the disclosure is not so limited. In embodiment, the software stored in the memory 32 may utilize location data obtained by the EM sensors 14a and/or 78 in combination with a series of 3D scenes from consecutive frames captured by the camera 74 of the second catheter 70 to create a more robust 3D reconstruction of the patient's airways than using the EM sensors 14a and/or 78 or the images captured by the camera 74 alone. In this manner, the location data obtained by the EM sensors 14a or 78 and the series of 3D scenes are correlated or otherwise registered to one another using the reference coordinate frame. It is envisioned that the software stored in the memory 32 may enhance or otherwise improve the 3D reconstruction formed from pre-operative images by adding additional information, such as the diameter of the airway in which the distal portion 72 of the second catheter 70 is disposed and the orientation of the airway relative to the orientation of the EM sensors 14a and/or 78. It is contemplated that the software stored in the memory 32 may identify a distance between the distal portion 72 of the second catheter 70 and the center C of the airway in which the distal portion 72 is disposed using illumination saturation (e.g., for example, asymmetric brightening). In embodiments, the software stored in the memory 32 may utilize any of the above described methods in combination with training a computer vision model or algorithm.

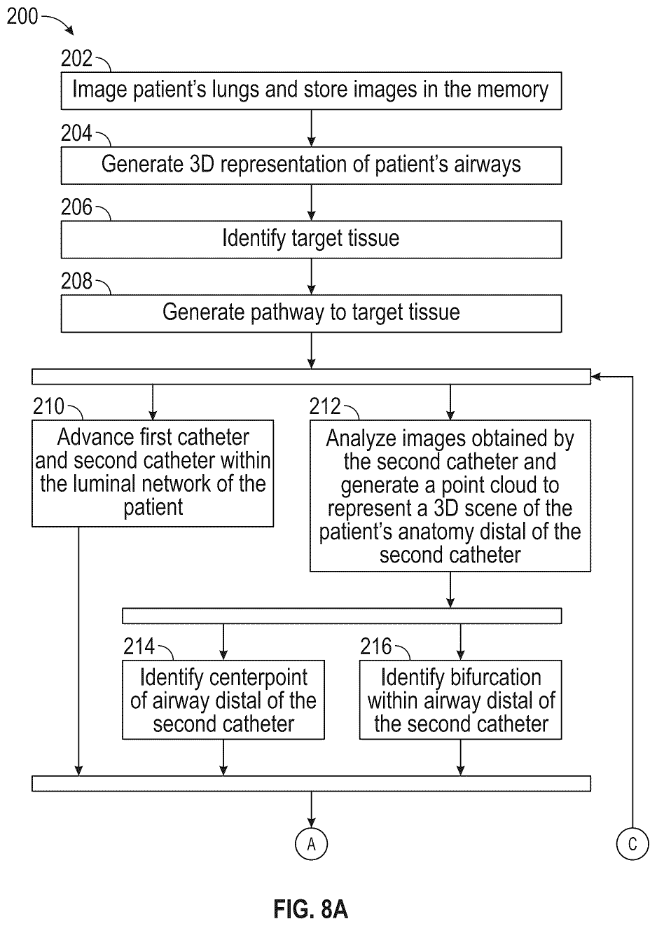

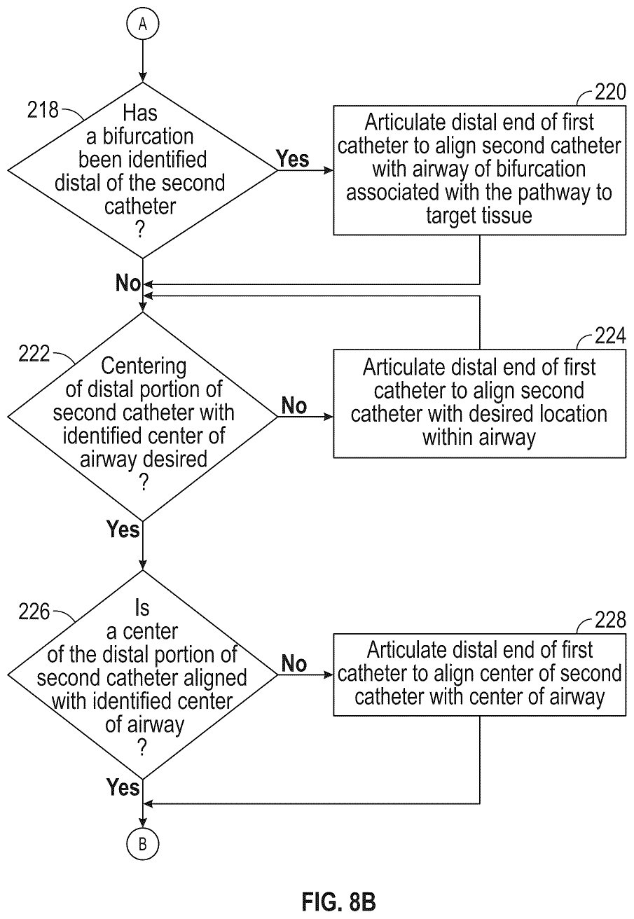

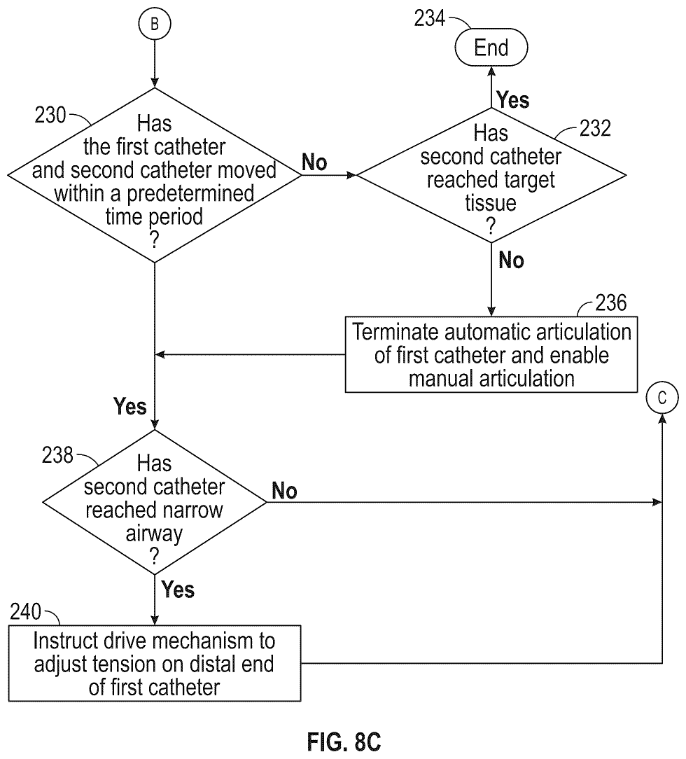

With reference to FIGS. 8A-8C, a method of performing a surgical procedure is illustrated and generally identified by reference numeral 200. Initially, at step 202, the patient P is imaged and the captured images are stored in the memory 32. In step 204, the software stored on the memory 32 generates a 3D representation of the patient P's airways. In step 206, target tissue TT is identified in the generated 3D representation of the patient P's airways and a pathway PW to the target tissue through the patient P's airways is generated in step 208. In step 210, the first catheter, which may be the sEWC 14, with the second catheter 70 advanced within and coupled thereto, is advanced within the luminal network of the patient P. In parallel, images of the patient P's anatomy distal of the second catheter 70 captured by the second catheter 70, which in embodiments may be captured by the camera 74, are analyzed and a 3D representation of a scene of the patient P's anatomy distal of the second catheter 70 is generated, which in embodiments, may be represented by a 3D point cloud. In step 214, a centerpoint C of the airway distal of the second catheter 70 is identified in parallel with identifying a bifurcation B within the airway distal of the second catheter 70 in step 216. In step 218, it is determined if a bifurcation B has been identified within the 3D representation of the scene of the patient P's anatomy distal of the second catheter 70. If a bifurcation B is identified, the software stored on the memory 32 instructs the drive mechanism 84 to articulate the distal end of the sEWC to align the distal portion 72 of the catheter with the airway of the bifurcation B associated with the pathway to the target tissue in step 220. If no bifurcation B is identified, or after the distal portion 72 of the second catheter 70 is aligned with the correct airway, in step 222, it is determined if centering the distal portion 72 of the second catheter 70 with the identified center C of the airway is desired. If no centering of the distal portion 72 of the second catheter 70 is desired, in step 224, the software stored in the memory 32 instructs the drive mechanism 84 to articulate the distal end of the sEWC 14 to align with or intersect a desired location within the airway and the method returns to step 222. If centering of the distal portion 72 of the second catheter 70 is desired, in step 226, it is determined if a center of the distal portion 72 of the second catheter 70 is aligned with the identified center C of the airway within the 3D representation of the scene of the patient P's anatomy distal of the second catheter 70. If it is determined that the center of the distal portion 72 of the second catheter 70 is not aligned with the center C of the airway, the software stored on the memory 32 instructs the drive mechanism 84 to articulate the distal end of the sEWC 14 and align the center of the distal portion 72 of the second catheter 70 with the center C of the airway or cause a pathway of the catheter 72 to intersect the center C of the airway in step 228. In step 230, if it is determined that the center of the distal portion 72 of the second catheter 70 is aligned with the center C of the airway, or after the drive mechanism has articulated the distal end of the sEWC 14 to align the second catheter 70 with the center C of the airway, it is determined if the sEWC 14 and second catheter 70 have moved within a predetermined time period. If it is determined that the sEWC 14 and second catheter 70 have not moved within a predetermined time period, it is determined whether the distal portion 72 of the second catheter 70 is disposed adjacent to the target tissue TT in step 232, and if so, the method ends in step 234. If it is determined that the distal portion 72 of the second catheter 70 is not disposed adjacent to the target tissue TT, the software stored in the memory 32 terminates automatic articulation of the distal end of the sEWC 14 and enables manual articulation of the distal end of the sEWC 14 in step 236. If in step 230 it is determined that the sEWC 14 and the second catheter 70 have moved within the predetermined time period, it is determined if the distal portion 72 of the second catheter 70 is disposed within a narrow airway in step 238. If it is determined that the distal portion 72 of the second catheter 70 is disposed within a narrow airway, in step 240, the software stored on the memory 32 instructs the drive mechanism 84 to adjust tension on the distal end of the sEWC 14 to permit walls of the airway to deflect the distal end of the sEWC 14 and minimize the potential of damaging or puncturing the walls of the airway. If it is determined that the distal portion 72 of the second catheter 70 is not disposed in a narrow airway, or after the drive mechanism 84 is instructed to relax the tension on the distal end of the sEWC 14, the method returns to steps 210 and 212. As can be appreciated, the above described method may be repeated as many times as necessary depending upon the procedure being performed.

Turning to FIG. 9, it is envisioned that the system 10 may include a robotic surgical system 600 having a drive mechanism 602 including a robotic arm 604 operably coupled to a base or cart 606, which may, in embodiments, be the workstation 20. The robotic arm 604 includes a cradle 608 that is configured to receive a portion of the sEWC 14. The sEWC 14 is coupled to the cradle 608 using any suitable means (e.g., for example, straps, mechanical fasteners, and/or couplings). It is envisioned that the robotic surgical system 600 may communicate with the sEWC 14 via electrical connection (e.g., for example, contacts and/or plugs) or may be in wireless communication with the sEWC 14 to control or otherwise effectuate movement of one or more motors (FIG. 2) disposed within the sEWC 14 and in embodiments, may receive images captured by a camera (not shown) associated with the sEWC 14 or the camera 74 of the second catheter 70. In this manner, it is contemplated that the robotic surgical system 600 may include a wireless communication system 610 operably coupled thereto such that the sEWC 14 and/or second catheter 70 may wirelessly communicate with the robotic surgical system 600 and/or the workstation 20 via Wi-Fi, Bluetooth®, for example. As can be appreciated, the robotic surgical system 600 may omit the electrical contacts altogether and may communicate with the sEWC 14 and/or second catheter 70 wirelessly or may utilize both electrical contacts and wireless communication. The wireless communication system 610 is substantially similar to the network interface 36 (FIG. 4) described hereinabove, and therefore, will not be described in detail herein in the interest of brevity. As indicated hereinabove, the robotic surgical system 600 and the workstation 20 may be one in the same, or in embodiments, may be widely distributed over multiple locations within the operating room. It is contemplated that the workstation 20 may be disposed in a separate location and the display 44 (FIGS. 1 and 4) may be an overhead monitor disposed within the operating room.

As indicated hereinabove, it is envisioned that the sEWC 14 may be manually or automatically articulated via cables or push wires 86, or for example, may be electronically operated via one or more buttons, joysticks, toggles, actuators (not shown) operably coupled to the drive mechanism 84 of the sEWC 14. As noted hereinabove, it is envisioned that the motors 88 of the drive mechanism 84 may not be disposed within the sEWC 14, and rather, the sEWC 14 may interface with motors 622 disposed within the cradle 608 of the robotic surgical system 600. In embodiments, the sEWC 14 may include a motor or motors 88 for controlling articulation of the distal end of the sEWC 14 in one plane (e.g., for example, left/null or right/null) and the drive mechanism 624 of the robotic surgical system 600 may include at least one motor 622 to effectuate the second axis of rotation and for axial motion. In this manner, the motor 88 of the sEWC 14 and the motors 622 of the robotic surgical system 600 cooperate to effectuate four-way articulation of the distal end of the sEWC 14 and effectuate rotation of the sEWC 14.

From the foregoing and with reference to the various figures, those skilled in the art will appreciate that certain modifications can be made to the disclosure without departing from the scope of the disclosure.

Although the description of computer-readable media contained herein refers to solid-state storage, it should be appreciated by those skilled in the art that computer-readable storage media can be any available media that can be accessed by the processor 30. That is, computer readable storage media may include non-transitory, volatile, and non-volatile, removable and non-removable media implemented in any method or technology for storage of information such as for example, computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the workstation 20.

The invention may be further described by reference to the following numbered paragraphs:

1. A surgical system, comprising:

an extended working channel (EWC), the EWC including a drive mechanism configured to articulate a distal end of the EWC;

a catheter operably coupled to the EWC, the catheter including a camera and an electromagnetic sensor; and

a workstation operably coupled to the EWC and the catheter, the workstation including processing means configured to:
determine a location and an orientation of the distal end of the EWC;
receive real-time images of the patient's anatomy from the camera of the catheter;
identify a centerpoint of a lumen within the received real-time images of the patient's anatomy;
determine if a center of the catheter is aligned with the identified centerpoint of the lumen;
articulate, if the catheter is misaligned with the identified centerpoint of the lumen, the distal end of the EWC using the drive mechanism to align the distal end of the EWC with the identified centerpoint of the lumen;
determine if the distal end of the EWC is located within an airway having a diameter that approximates an outer diameter of the catheter; and
instruct, if the distal end of the EWC is located within an airway having a diameter that approximates the outer diameter of the catheter, the drive mechanism to reduce tension on the distal end of the EWC to permit the distal end of the EWC to deflect when contacting walls of the lumen.

2. The system according to paragraph 1, wherein the processing means is configured to generate a three-dimensional (3D) representation of a scene distal of the camera of the catheter.

3. The system according to paragraph 2, wherein the processing means is configured to generate the 3D representation of the scene using pixel-wise depth estimation; and

identify a centerpoint of the lumen using data resulting from the pixel-wise depth estimation.

4. The system according to paragraph 1, wherein the processing means is configured to generate a 3D model of the airways of the patient using pre-procedure images of the patient's anatomy.

5. The system according to paragraph 4, wherein the processing means is configured to generate a pathway through the airways of the patient to target tissue identified in the generated 3D model.

6. The system according to paragraph 5, wherein the processing means is configured to identify a bifurcation within the received real-time images of the patient's anatomy.

7. The system according to paragraph 6, wherein the processing means is configured to articulate the distal end of the EWC using the drive mechanism to align the distal end of the EWC with a lumen of the bifurcation associated with the pathway to the target tissue.

8. The system according to paragraph 1, wherein the processing means is configured to determine if a position of the distal end of the EWC within the patient's airways has changed within a predetermined time period; and

disable the drive mechanism if it is determined that the position of the distal end of the EWC has not changed within the predetermined time period.

9. A method of operating a surgical system, the method comprising:

determining a pose of a distal end of an extended working channel (EWC);

receiving real-time images of a patient's anatomy from a camera coupled to a catheter, the catheter received within a portion of the EWC;

identifying a centerpoint of a lumen within the received real-time images of the patient's anatomy;

determining if a center of the catheter is aligned with the identified centerpoint of the lumen;

articulating, if the catheter is misaligned with the identified centerpoint of the lumen, the distal end of the EWC using a drive mechanism to align the distal end of the EWC with the identified centerpoint of the lumen, wherein the drive mechanism is operably coupled to the EWC;

determining if the distal end of the EWC is located within an airway having a diameter that approximates the outer diameter of the catheter; and

instructing, if the distal end of the EWC is located within an airway having a diameter that approximates the outer diameter of the catheter, the drive mechanism to reduce tension on the distal end of the EWC to permit the distal end of the EWC to deflect when contacting walls of the lumen.

10. The method according to paragraph 9, further comprising generating a three-dimensional (3D) representation of a scene of the camera of the catheter.

11. The method according to paragraph 10, wherein the 3D representation of the scene is generated using pixel-wise depth estimation.

12. The method according to paragraph 9, further comprising determining if a position of the distal end of the EWC within the patient's airways has changed within a predetermined time period; and

disabling the drive mechanism if it is determined that the position of the distal end of the EWC has not changed within the predetermined time period.

13. A method of operating a surgical device, the method comprising:

determining a pose of a distal end of an extended working channel (EWC) using an inertial measurement unit coupled to the EWC;

receiving real-time images of a patient's anatomy from a camera coupled to a catheter, wherein the catheter is received within a portion of the EWC;

articulating the distal end of the EWC, using a drive mechanism coupled to the EWC, to align a distal portion of the catheter with a lumen of a bifurcation identified within the received real-time images; and

instructing the drive mechanism to reduce tension on the distal end of the EWC if a determined outer dimension of the lumen approximates an outer dimension of the catheter to permit the distal end of the EWC to deflect when contacting walls of the lumen.

14. The method according to paragraph 13, further comprising generating a three-dimensional representation of a scene distal of the camera of the catheter using pixel-wise depth estimation.

15. The method according to paragraph 13, further comprising determining if a position of the distal end of the EWC within the patient's airways has changed within a predetermined time period; and

disabling the drive mechanism if it is determined that the position of the distal end of the EWC has not changed within the predetermined time period.

What is claimed is:

1. A system for performing a surgical procedure, comprising:
an extended working channel (EWC), the EWC including a drive mechanism configured to articulate a distal end of the EWC;
a catheter operably coupled to the EWC, the catheter including a camera and an electromagnetic (EM) sensor; and
a workstation operably coupled to the EWC and the catheter, the workstation including a memory and a processor, the memory storing instructions, which when executed by the processor cause the processor to:
determine a location and an orientation of the distal end of the EWC;
receive real-time images of the patient's anatomy from the camera of the catheter;
identify a centerpoint of a lumen within the received real-time images of the patient's anatomy;
determine if a center of the catheter is aligned with the identified centerpoint of the lumen;
articulate, if the catheter is misaligned with the identified centerpoint of the lumen, the distal end of the EWC using the drive mechanism to align the distal end of the EWC with the identified centerpoint of the lumen;
determine if the distal end of the EWC is located within an airway having a diameter that approximates an outer diameter of the catheter; and
instruct, if the distal end of the EWC is located within an airway having a diameter that approximates the outer diameter of the catheter, the drive mechanism to reduce tension on the distal end of the EWC to permit the distal end of the EWC to deflect when contacting walls of the lumen.

2. The system according to claim 1, further comprising the memory storing thereon further instructions, which when executed by the processor cause the processor to generate a three-dimensional (3D) representation of a scene distal of the camera of the catheter.

3. The system according to claim 2, further comprising the memory storing thereon further instructions, which when executed by the processor cause the processor to generate the 3D representation of the scene using pixel-wise depth estimation.

4. The system according to claim 3, further comprising the memory storing thereon further instructions, which when executed by the processor cause the processor to identify the centerpoint of the lumen using data resulting from the pixel-wise depth estimation.

5. The system according to claim 1, further comprising the memory storing thereon further instructions, which when executed by the processor cause the processor to generate a 3D model of the airways of the patient using pre-procedure images of the patient's anatomy.

6. The system according to claim 5, further comprising the memory storing thereon further instructions, which when executed by the processor cause the processor to generate a pathway through the airways of the patient to target tissue identified in the generated 3D model.

7. The system according to claim 6, further comprising the memory storing thereon further instructions, which when executed by the processor cause the processor to identify a bifurcation within the received real-time images of the patient's anatomy.

8. The system according to claim 7, further comprising the memory storing thereon further instructions, which when executed by the processor cause the processor to articulate the distal end of the EWC using the drive mechanism to align the distal end of the EWC with a lumen of the bifurcation associated with the pathway to the target tissue.

9. The system according to claim 1, further comprising the memory storing thereon further instructions, which when executed by the processor cause the processor to determine if a position of the distal end of the EWC within the patient's airways has changed within a predetermined time period.

10. The system according to claim 9, further comprising the memory storing thereon further instructions, which when executed by the processor cause the processor to disable the drive mechanism if it is determined that the position of the distal end of the EWC has not changed within the predetermined time period.

11. A method for navigating a surgical device to an area of interest, the method comprising:
determining a pose of a distal end of an extended working channel (EWC);
receiving real-time images of a patient's anatomy from a camera coupled to a catheter, the catheter received within a portion of the EWC;
identifying a centerpoint of a lumen within the received real-time images of the patient's anatomy;
determining if a center of the catheter is aligned with the identified centerpoint of the lumen;
articulating, if the catheter is misaligned with the identified centerpoint of the lumen, the distal end of the EWC using a drive mechanism to align the distal end of the EWC with the identified centerpoint of the lumen, wherein the drive mechanism is operably coupled to the EWC;
determining if the distal end of the EWC is located within an airway having a diameter that approximates an outer diameter of the catheter; and
instructing, if the distal end of the EWC is located within an airway having a diameter that approximates the outer diameter of the catheter, the drive mechanism to reduce tension on the distal end of the EWC to permit the distal end of the EWC to deflect when contacting walls of the lumen.

12. The method according to claim 10, further comprising generating a three-dimensional (3D) representation of a scene distal of the camera of the catheter.

13. The method according to claim 12, wherein the 3D representation of the scene is generated using pixel-wise depth estimation.

14. The method according to claim 11, further comprising determining if a position of the distal end of the EWC within the patient's airways has changed within a predetermined time period.

15. The method according to claim 14, further comprising disabling the drive mechanism if it is determined that the position of the distal end of the EWC has not changed within the predetermined time period.

16. A method of navigating a surgical device to an area of interest, the method comprising:
determining a pose of a distal end of an extended working channel (EWC) using an inertial measurement unit coupled to the EWC;
receiving real-time images of a patient's anatomy from a camera coupled to a catheter, wherein the catheter is received within a portion of the EWC;
articulating the distal end of the EWC, using a drive mechanism coupled to the EWC, to align a distal portion of the catheter with a lumen of a bifurcation identified within the received real-time images; and
instructing the drive mechanism to reduce tension on the distal end of the EWC if a determined outer dimension of the lumen approximates an outer dimension of the catheter to permit the distal end of the EWC to deflect when contacting walls of the lumen.

17. The method according to claim 16, further comprising generating a three-dimensional (3D) representation of a scene distal of the camera of the catheter.

18. The method according to claim 17, wherein the 3D representation of the scene is generated using pixel-wise depth estimation.

19. The method according to claim 16, further comprising determining if a position of the distal end of the EWC within the patient's airways has changed within a predetermined time period.

20. The method according to claim 19, further comprising disabling the drive mechanism if it is determined that the position of the distal end of the EWC has not changed within the predetermined time period.
